(19) United States
(12) Patent Application Publication
(10) Pub. No.: US 2026/0140047 A1
(43) Pub. Date: May 21, 2026

(54) GEOLOGICAL SKETCHING METHOD FOR TUNNEL FACES BASED ON IMAGE SPECTRUM TECHNOLOGY, AND SYSTEM THEREOF

(71) Applicant: SHANDONG UNIVERSITY, Jinan, Shandong (CN)

(72) Inventors: Zhenhao XU, Jinan (CN); Peng LIN, Jinan (CN); Shan Li, Jinan (CN); Huihui XIE, Jinan (CN); Heng SHI, Jinan (CN); Ruiyang MA, Jinan (CN); Yingchun HE, Jinan (CN); Ruiqi SHAO, Jinan (CN); Hang XIANG, Jinan (CN); Shucai LI, Jinan (CN)

(73) Assignee: SHANDONG UNIVERSITY, Jinan, Shandong (CN)

(21) Appl. No.: 19/118,860

(22) PCT Filed: Oct. 31, 2023
(86) PCT No.: PCT/CN2023/128809

§ 371 Date: Apr. 7, 2025

(30) Foreign Application Priority Data
Oct. 19, 2022 (CN) 202211281307.4

(51) Int. Cl. G01N 21/31; G01N 21/95; G01N 33/24; G06T 11/10; G06T 11/23; G06V 10/26; G06V 10/54; G06V 10/56; G06V 10/764; G06V 20/70

(52) U.S. Cl. CPC G01N 21/31; G01N 21/95; G01N 33/24; G06T 11/10; G06T 11/23; G06V 10/273; G06V 10/54; G06V 10/56; G06V 10/764; G06V 20/70

(57) ABSTRACT

An image spectrum technology-based geological sketching method, including: based on collected image-spectrum data information of a tunnel face, extracting mineral end members and spectrum to determine types and contents of minerals, extracting texture features and feature waveband spectrums to determine stratum lithology, extracting crack features and identifying crack fillers to obtain a crack identification result; obtaining degrees of crushing of surrounding rocks according to numbers and relative areas of cracks; analyzing weathering variation proportions of minerals according to stratum lithology and mineral analysis result for surrounding rocks, acquiring color differences between different degrees of weathering according to spectral color difference identification results, obtaining degrees of weathering of the surrounding rocks; identifying water outflow form and water outflow position of tunnel face to be detected; and obtaining a geological sketch map for the tunnel face by performing labeling and summarization by using different labeling symbols according to the results.

The present disclosure claims the priority of the Chinese Patent Application 202211281307.4 filed to China National Intellectual Property Administration on Oct. 19, 2022, and entitled “GEOLOGICAL SKETCHING METHOD FOR TUNNEL FACES BASED ON IMAGE SPECTRUM TECHNOLOGY, AND SYSTEM THEREOF”, which is incorporated herein by reference in its entirety.

### TECHNICAL FIELD

The present disclosure relates to the technical field of intelligent geological logging of a tunnel, and particularly relates to a geological sketching method for tunnel faces based on an image spectrum technology, and system thereof.

### BACKGROUND

The description in this section merely provides background information related to the present disclosure and does not necessarily constitute the related art.

Geological conditions of tunnel construction are complex, unfavorable geologic bodies such as fault fracture zones, alteration zones, and karst have relatively great concealment, and it is an important means of performing geological logging in a tunnel construction process to master the unfavorable geological conditions along of a tunnel. Through geological logging, we can quickly, accurately and comprehensively know the engineering geological and hydrogeological conditions of an excavated section of a tunnel, so as to check, correct and perfect geological data designed in early investigation, and a geological basis can also be provided for tunnel advanced geological prediction and tunnel construction solution optimization. By utilizing geological sketch information feedback of a tunnel face in tunnel construction, objectives of dynamic feedback design and efficient construction can be achieved, and at the same time, the surrounding rock information of the tunnel face is collected, consolidated and systematically analyzed for advanced geological prediction of the tunnel under different geological conditions.

A conventional geological sketching method for a tunnel has defects of time consumption, great subjectivity, and severe rely on experience and carefulness of geological workers. The logging contents are not comprehensive enough, erroneous judgment and missed judgment phenomena easily occur, and certain risks exist. Working personnel perform checking with security risks brought by intimate contact with a tunnel face.

In addition, the inventor discovers that most of existing intelligent geological logging of a tunnel face relies on collection of shot pictures of the tunnel face, however, the mineral content information of rocks, geological conditions with unobvious lithofacies characteristics and the like cannot be accurately identified during acquisition of the geological logging information based on image deep learning technology.

Information such as composition of surrounding rocks can be quantitatively acquired by using spectrum technology, errors caused by artificial analysis are reduced, and the precision of the geological sketch is greatly improved. However, the existing spectrum technology has disadvantages of small test range, need of handheld touch operation on site, low efficiency, etc.

### SUMMARY

In order to solve the above problems, the present disclosure provides a geological sketching method for tunnel faces based on an image spectrum technology, and system thereof. Based on the image spectrum technology, image information and spectral information of the tunnel face are acquired, and the image information and the spectral information are merged to perform analysis on a stratum feature, a geological structure and hydrogeology, so as to realize the digital geological sketch of the tunnel face.

In order to achieve the above objectives, the present disclosure adopts the following technical solution:
according to a first aspect, the present disclosure provides a geological sketching method for tunnel faces based on an image spectrum technology, including:
acquiring image information and spectral information of a tunnel face to be subjected to detection;
performing mineral end member extraction and spectral unmixing on the spectral information by using a mixed pixel decomposition method, identifying the types and contents of minerals, and determining space distribution of different minerals;
extracting a spectrum feature vector according to the spectral information, extracting an image feature vector according to the image information, and determining the stratum lithology according to the spectrum feature vector and the image feature vector by using a trained classifier;
extracting a crack feature according to the image information, identifying a crack filler according to the spectral information, and obtaining a crack identification result according to the crack feature and the crack filler;
acquiring the number and relative areas of cracks according to the crack identification result so as to obtain the degree of crushing of surrounding rocks, obtaining the weathering variation proportion of the minerals according to the stratum lithology and comparison with existing types and contents of minerals, obtaining a color difference classification result of different weathering types and regions according to color features of different regions, and obtaining the degree of weathering of the surrounding rocks according to the degree of crushing of the surrounding rocks, the weathering variation proportion of the minerals and the color difference classification result;
according to the image information, identifying a water outflow form and a water outflow position of a tunnel face to be subjected to detection; and
integrating the stratum lithology, the space distribution and contents of the minerals and the degree of weathering of the surrounding rocks into a first geological sketch map, and integrating the crack identification result, the water outflow form and the water outflow position into a second geological sketch map, so as to complete the geological sketch on the tunnel face.

As an optional implementation, a process of identifying the types and contents of minerals includes:
extracting a mineral end member, and performing spectrum matching on the mineral end member and the corresponding spectrum with a prebuilt reference wave spectrum, so as to determine the types of the minerals; and
decomposing the identified types of the minerals as the end member to obtain the percentage contents of each type of minerals, labeling a mineral association, the mineral end member and the contents thereof according to each pixel, and performing weighting processing on the weight of each type of minerals obtained by unmixing each pixel end member and the pixel point occupied by the minerals to obtain the mineral content.

As an optional implementation, the image information and the spectral information of the tunnel face to be subjected to detection are rasterized, and a spectrum feature vector is extracted after the equalization processing on the spectral information in grids; and
the image feature vector includes texture features of a feature waveband, and a process of extracting the texture feature includes: obtaining a two-dimensional gray level image after performing grey processing on the image information, extracting the texture feature of the two-dimensional gray level image by using a grey-level co-occurrence matrix method, and calculating feature parameters in four directions of 0°, 45°, 90°, and 135°.

As an optional implementation, the crack identification result includes positions of the cracks, attitudes of the cracks, apertures of the cracks and crack fillers; and
a crack identification process includes: distinguishing cracks from a background by using an image segmentation method, removing other elements except for the cracks, and acquiring crack skeletons and crack profiles; and performing rasterization processing by using the crack skeletons as a center and the crack profiles as a boundary, and identifying the crack filler according to the spectral information in the grids.

As an optional implementation, a process of identifying the degree of weathering of the surrounding rocks includes:
according to the number and relative areas of the cracks, using a pretrained surrounding rock crushing degree classification model to obtain a classification result for the degree of crushing of the surrounding rocks;
analyzing whether there is a mineral composition change or not according to the stratum lithology and comparison with existing types and contents of minerals, and using a pretrained mineral weathering variation proportion model to obtain a mineral weathering variation proportion;
expressing surface color differences of regions of different weathering types and weathering degrees by using spectral color differences according to the color features of different regions; and
respectively weighting the degree of crushing of the surrounding rocks, the mineral weathering variation proportion and the color difference classification result to obtain the degree of weathering of the surrounding rocks.

As an optional implementation, a water outflow form of the tunnel face is obtained according to the image information by using a trained water outflow image identification model, and a water outflow position is determined;
the water outflow image identification model is obtained by training a built network framework by using the tunnel face images at different water outflow types according to the relationship between the tunnel face water outflow image and the rock water outflow condition of the surrounding rocks of the tunnel face; and
the water outflow form includes no water outflow sign, water seepage, water dripping, threadlike water flowing, water spouting, and water gushing.

As an optional implementation, a tunnel face geological sketching process includes:
labeling the types of the minerals by using different colors;
labeling the contents of the minerals by using content contour lines;
labeling the stratum lithology by using different lithology legends and symbols;
labeling the degree of weathering of the surrounding rocks for a region with surrounding rock weathering;
labeling the positions, the attitudes and the apertures of the cracks by using lines;
labeling the type of the crack filler by using different colors; and
labeling the water outflow form by using lines and the like.

According to a second aspect, the present disclosure provides a geological sketching system for tunnel faces based on an image spectrum technology, including:
an image and spectral data body acquiring module, configured to acquire image information and spectral information of a tunnel face to be subjected to detection;
a mineral composition identification module, configured to perform mineral end member extraction and spectral unmixing on the spectral information by using a mixed pixel decomposition method, identify the types and contents of minerals, and determine space distribution of different minerals;
a stratum lithology identification module, configured to extract a spectrum feature vector according to the spectral information, extract an image feature vector according to the image information, and determine the stratum lithology according to the spectrum feature vector and the image feature vector by using a trained classifier;
a crack identification module, configured to extract a crack feature according to the image information, identify a crack filler according to the spectral information, and obtain a crack identification result according to the crack feature and the crack filler;
a weathering degree identification module, configured to acquire the number and relative areas of cracks according to the crack identification result so as to obtain the degree of crushing of surrounding rocks, obtain the weathering variation proportion of the minerals according to the stratum lithology and comparison with existing types and contents of minerals, obtain a color difference classification result of different weathering types and regions according to color features of different regions, and obtain the degree of weathering of the surrounding rocks according to the degree of crushing of the surrounding rocks, the weathering variation proportion of the minerals and the color difference classification result;
a water outflow identification module, configured to identify a water outflow form and a water outflow position of the tunnel face to be subjected to detection according to the image information; and
a geological sketching module, configured to integrate the stratum lithology, the space distribution and contents of the minerals and the degree of weathering of the surrounding rocks into a first geological sketch map, and integrate the crack identification result, the water outflow form and the water outflow position into a second geological sketch map, so as to complete the geological sketch on the tunnel face.

According to a third aspect, the present disclosure provides an electronic device, including a memory, a processor, and computer instructions stored in the memory and running on the processor. When the computer instructions are run by the processor, the method according to the first aspect is implemented.

According to a fourth aspect, the present disclosure provides a computer readable storage medium configured to store computer instructions. When the computer instructions are executed by the processor, the method according to the first aspect is implemented.

Compared with the related art, the present disclosure has the following beneficial effects:

1. The geological sketching method for tunnel faces based on an image spectrum technology, and system thereof provided by the present disclosure adopt a non-contact and in-situ measurement manner, and belong to an important technical means of fast observing material composition in a large area. A three-dimensional data body may be acquired by once shooting, one-dimensional spectral information of tens to hundreds of continuous wavebands may be collected at the same time when two-dimensional space information of a tunnel face to be subjected to detection is acquired, and the advantages of abundant volume of acquired data and unified image and spectrum are realized.

2. The present disclosure provides a geological sketching method for tunnel faces based on an image spectrum technology, and system thereof. The image information of the tunnel face to be subjected to detection and the spectral information of each pixel point are acquired based on the image spectrum technology, the image information and the spectral information are integrated to perform multi-source information merging identification on the stratum lithology, mixed pixels are unmixed to identify the mineral end member, weighting processing is performed to analyze the contents of the minerals, the distribution of the cracks and the attitudes of the cracks are identified by using the image information, the crack filling minerals are detected through the spectrum, the degree of weathering of the surrounding rocks is comprehensively judged through the degree of crushing, the mineral weathering variation proportion and the color difference, and the hydrogeology such as the water outflow positions and the water outflow form is analyzed to realize the quantitative analysis on the geological sketch of the tunnel face.

3. The geological sketching method for tunnel faces based on an image spectrum technology, and system thereof provided by the present disclosure replace a traditional geological sketching method. With the help of the image and spectral information of the image spectrum technology, the problems of qualitative analysis at a subjective level, incomprehensive logging contents and easy occurrence of erroneous judgment and missed judgment phenomena are greatly solved, the precision and efficiency of the geological logging are greatly improved, and the intelligent geological sketch of the tunnel face is realized.

4. According to the geological sketching method for tunnel faces based on an image spectrum technology, and system thereof provided by the present disclosure, an artificial intelligence and data mining measure is used for building data processing and prediction identification models for engineering geological information and hydrogeological information, and the span from the qualitative analysis at the subjective level to the intelligent quantitative analysis is realized.

The advantages in additional aspects of the present disclosure will be set forth in part in the description below, parts of which will become apparent from the description below, or will be understood by the practice of the present disclosure.

### BRIEF DESCRIPTION OF THE DRAWINGS

The accompanying drawings of the specification constituting a part of the present disclosure are used to provide a further understanding of the present disclosure. The exemplary embodiments of the present disclosure and descriptions thereof are used to explain the present disclosure, and do not constitute an improper limitation of the present disclosure.

FIG. 1 is a schematic flow chart of a geological sketching method for tunnel faces based on an image spectrum technology provided by Example 1 of the present disclosure;

FIG. 2 is a schematic diagram of an image spectrum technology-based imaging mode provided by Example 1 of the present disclosure;

FIG. 3A is a geological sketch map provided by Example 1 of the present disclosure; and

FIG. 3B is another one geological sketch maps provided by Example 1 of the present disclosure.

### DETAILED DESCRIPTION

The present disclosure will be further illustrated hereafter in combination with accompanying drawings and embodiments.

It should be noted that, the following detailed descriptions are all exemplary, and are intended to provide further descriptions of the present disclosure. Unless otherwise specified, all technical and scientific terms used herein have the same meanings as those usually understood by a person of ordinary skill in the art to which the present disclosure belongs.

It should be noted that the terms used herein are merely used for describing specific implementations, and are not intended to limit exemplary implementations of the present disclosure. As used herein, the singular form is also intended to include the plural form unless specifically stated otherwise, and in addition, it should also be understood that the terms “include”, “have”, and any variation thereof are intended to cover non-exclusive inclusions, for example, processes, methods, systems, products or devices including a series of steps or units do not need to be limited to those steps or units that are clearly listed, but may include other steps or units which are not clearly listed or are inherent to those processes, methods, products or devices.

The examples in the present disclosure and features in the embodiments may be mutually combined in case that no conflict occurs.

### Example 1

The present example provides a geological sketching method for tunnel faces based on an image spectrum technology. The image spectrum technology is a nondestructive in situ detection method capable of acquiring image information and spectral information at the same time, and through comprehensive analysis by merging the image and spectral information, the geological logging on the tunnel face may be comprehensively and precisely performed.

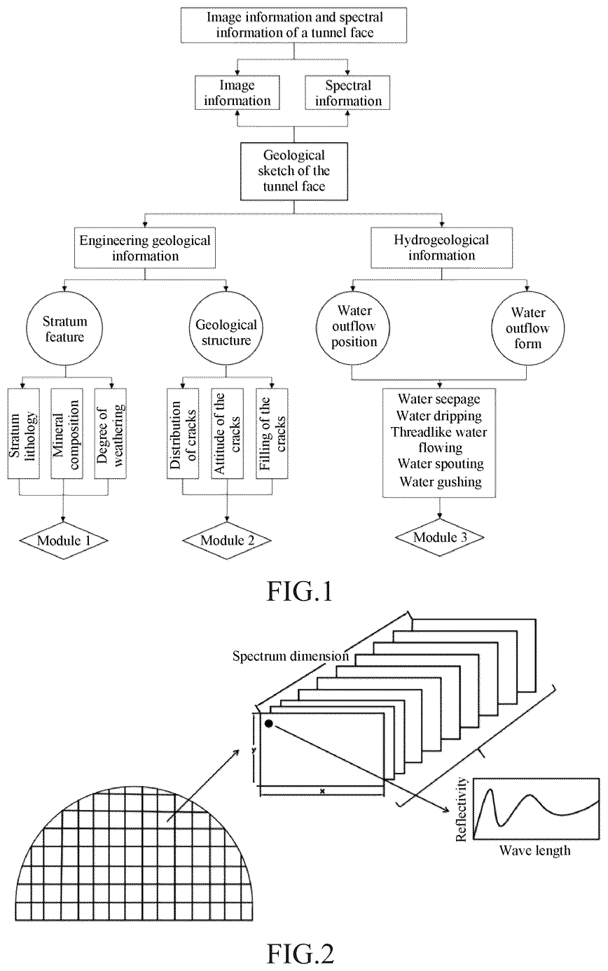

As shown in FIG. 1, the method specifically includes:
a three-dimensional image and spectral data body of a tunnel face to be subjected to detection, including image information and spectral information is acquired;
according to the spectral information of each pixel point, mineral end member extraction and spectral unmixing are performed by using a mixed pixel decomposition method, the types and contents of minerals are identified, and space distribution of different minerals is determined;
rasterization processing is performed on the image information and the spectral information of the tunnel face to be subjected to detection;
the spectral information in each grid is subjected to equalization processing, then, a spectrum feature vector is extracted, an image feature vector is extracted for the image information, the spectrum feature vector and the image feature vector are normalized, and then, the stratum lithology is determined by using a trained classifier;
for each grid, a crack feature is extracted according to the image information, a crack filler is identified according to the spectral information, a crack identification result is obtained according to the crack feature and the crack filler, and the crack identification result includes positions of the cracks, attitudes of the cracks, apertures of the cracks and crack fillers;
the number and relative areas of cracks are acquired according to the crack identification result so as to obtain the degree of crushing of surrounding rocks, the weathering variation proportion of the minerals is obtained according to the stratum lithology and comparison with existing types and contents of minerals of the surrounding rocks, a color difference classification result of different weathering types and regions is obtained according to color features of different regions, and the degree of weathering of the surrounding rocks is obtained according to the degree of crushing of the surrounding rocks, the weathering variation proportion of the minerals and the color difference classification result;
according to the image information, a water outflow form and a water outflow position of a tunnel face to be subjected to detection are identified; and
the stratum lithology, the space distribution and contents of the minerals and the degree of weathering of the surrounding rocks are integrated into a first geological sketch map, and the crack identification result, the water outflow form and the water outflow position are integrated into a second geological sketch map, so as to complete the geological sketch on the tunnel face, where different colors and content contour lines are used for labeling according to the types and contents of the minerals, the stratum lithology is labeled by different legends and symbols, a region labeling form is adopted for the degree of weathering of the surrounding rocks, and finally, the stratum lithology, the space distribution and contents of the minerals, and the degree of weathering of the surrounding rocks are summarized to obtain a first geological sketch map; and similarly, the crack identification result, the water outflow form and the water outflow positions are summarized to obtain a second geological sketch map.

In the present example, the tunnel face to be subjected to detection is subjected to image and spectral information collection by using an imaging spectrometer, FIG. 2 shows an image spectrum technology-based imaging manner, a non-contact non-destructive in situ scanning manner is used, a three-dimensional image and spectral data body may be acquired by once shooting, the three-dimensional image and spectral data body includes image information and spectral information, one-dimensional spectral information of tens to hundreds of continuous wavebands may be collected at the same time when two-dimensional space information of a tunnel face to be subjected to detection is acquired, and the advantages of abundant volume of acquired data and unified image and spectrum are realized.

The image information of the tunnel face to be subjected to detection and the spectral information of each pixel point are acquired based on the image spectrum technology, the image information and the spectral information are merged to analyze the stratum feature such as the stratum lithology, the mineral composition and the degree of weathering, analyze the geological structure of the distribution of the cracks, the attitudes of the cracks and the filling of the cracks, and analyze the hydrogeology such as the water outflow position and the water outflow form, so that the digital geological sketch on the tunnel face is realized, and the problems of strong subjectivity, incomprehensiveness and the like of conventional tunnel face sketching are solved.

In the present example, after the image information and the spectral information of the tunnel face to be subjected to detection are acquired, the image information and the spectral information are subjected to preprocessing. The preprocessing includes denoising, contrast enhancement and target interested space extraction. Stratum feature analysis, geological structure analysis and hydrological feature analysis are respectively performed according to the preprocessed image and spectral information, so as to extract the engineering geological information and hydrogeological information of the tunnel face to be subjected to detection. The engineering geological information includes a stratum feature and a geological structure. The stratum feature includes stratum lithology, a mineral composition, and a degree of weathering. The geological structure includes distribution of cracks, attitudes of the cracks, apertures of the cracks and crack fillers. The hydrogeological information includes a water outflow position and a water outflow form.

In the present example, the mineral spectral unmixing process includes mineral end member extraction, spectrum matching, mineral type identification, and mineral content inversion. Firstly, the spectral information of a mineral detection point is extracted by means of a mixed pixel decomposition method. Then, a mineral association, the mineral end member and the contents thereof are labeled according to each pixel of the image, and weighting processing is performed on the weight of each type of minerals obtained by unmixing each pixel end member and the pixel point occupied by the minerals to obtain the mineral content of a research region.

### Specifically:

according to precision requirements of geological logging of different tunnels, a distribution distance of mineral detection points is selected, and spectral information of the corresponding detection point is extracted.

Water-vapor absorption removal and denoising smoothing processing are performed on the spectral information, so as to remove various accidental errors. This is because in a process of performing wave spectrum measurement on the tunnel face, the influence of water vapor and atmosphere is inevitable, and the wave spectrum has fluctuation to different degrees in the atmosphere or water-vapor absorption bands, so the analysis and removal are needed. Through denoising smoothing, the influence of noise may be reduced to a certain degree commonly used denoising smoothing methods include a moving average method, a static average method and a Fourier series approximation method.

In the present example, a hybrid pixel decomposition method is used as an example. The mixed pixel decomposition method is used for performing surrounding rock mineral type identification, content quantitative analysis, and weighting processing.

The mineral end member and spectrum extraction is performed by using the mixed pixel decomposition method according to the processed spectral information. The method includes the following steps: the mineral end member extraction, the mineral type identification, and the abundance (mineral content) inversion.
the mineral end member extraction may be image-based end member extraction, such as a pure pixel index (PPI) method and an iterative error analysis (IEA) method.

The mineral end member information is extracted by using the above method. Then, spectrum matching is performed on the mineral end member and the corresponding spectrum with a prebuilt reference wave spectrum, and the types of the minerals of the surrounding rocks are determined through similarity calculation.

The spectrum matching method includes methods such as distance similarity measurement, angle similarity measurement (spectrum angle), spectrum correlation coefficient, and spectrum binary encoding. By taking a spectrum angle measuring method as an example, an included angle value between an unknown mineral end member spectrum and a reference wave spectrum is calculated to determine their similarity, and the unknown mineral end member spectrum is classified according to a similarity threshold.

The mineral content inversion includes: firstly, the identified types of the minerals of the surrounding rocks are decomposed as the end member to obtain the percentage contents of each type of minerals of the surrounding rocks, then, a mineral association, the mineral end member and the contents thereof are labeled according to each pixel of the image, and weighting processing is performed on the weight of each type of minerals obtained by unmixing each pixel end member and the pixel point occupied by the minerals to obtain the mineral content of the research region.
the digitization conditions of different minerals are weighted according to region contents:

$$Content = \frac{\sum Weight of mixed end member * number of pixel points}{Total number of pixels}$$

In the present example, the detected compositions and contents of the minerals of the surrounding rocks may further be used for showing the change trend of the contents of the minerals in a form of a brief dot and line schematic diagram. In a summarized geological sketch map, the contents of the minerals are displayed through the content contour lines, and the spatial distribution condition of the minerals on the tunnel face may be further shown in a mineral mapping form.

In the present example, the image information and the spectral information of the tunnel face to be subjected to detection are rasterized, a spectrum feature vector is extracted after the equalization processing on the spectral information in each grid, and one spectral curve is extracted from one grid. Then, an image feature vector of the image information in the grids is extracted, the spectrum feature vector and the image feature vector are normalized, and next, the stratum lithology is identified by using a trained classifier.

Specifically:
the tunnel face is meshed, and image information and spectral information of each grid are extracted;
the processing on the spectral information includes spectrum preprocessing and feature waveband spectrum extraction; and
the spectrum preprocessing method includes S-G convolutional smoothing, baseline correction, standard normal variable transformation, first-order derivative, second-order derivative and trend removal.

Through the S-G convolution smoothing, the smoothness of the spectrum may be effectively improved, and the high-frequency noise interference may be reduced. The standard normal variable transformation is mainly to reduce the influence of nonuniform size of solid particles, object surface scattering and optical path transformation on spectrum data. The trend removal is used for dealing with a problem of diffuse reflection spectrum baseline shift, and is generally used in combination with the standard normal variable transformation. The first-order derivative and second-order derivative methods are used for background interference elimination and baseline correction, so as to improve resolution and sensitivity.

The feature waveband spectrum selection methods include principal component analysis, minimum noise separation, and continuous projection algorithm and boundary decision.

The image feature vector extracted from the image information includes texture features of a feature waveband. A process of extracting the texture feature includes: a two-dimensional gray level image is obtained after the grey processing on the image information, the texture feature of the two-dimensional gray level image is extracted by using a grey-level co-occurrence matrix method, and feature parameters, such as energy, entropy, inertia moment and correlation are calculated in four directions of 0°, 45°, 90°, and 135°.

After the feature waveband spectrum and the texture feature are subjected to normalizing processing, and the stratum lithology is determined by using a trained classifier; and the classifier includes a back-propagation neural network, a linear discriminant analyzer, an extreme learning machine, a random forest and a nonlinear partial least square support vector machine.

In the present example, the crack skeletons and the boundary profiles are identified according to the image information, and the features of the cracks such as the positions of the cracks, the attitudes of the cracks and the apertures of the cracks are labeled on the sketch map. Then, rasterization processing is performed by using the crack skeletons and the boundary profiles as the reference, so that the grids cover the mineral filler positions, the spectral information in each grid is extracted, the crack filler in the crack grid is identified according to the spectral information, and the crack feature and the crack filler minerals are supplemented to complete the crack information.

Specifically:
the cracks are distinguished from a background by using an image segmentation method. The image segmentation method includes a threshold segmentation method, a boundary-based segmentation method, a region-based segmentation method and a segmentation method combined with a particular theoretical tool.

Other elements except for the cracks in the image are removed, and the crack skeletons and crack profiles are extracted.

A binary image skeleton thinning algorithm, such as a Zhang Suen thinning algorithm and a Thinning-Algorithm, including image skeleton thinning and pruning algorithm collateral removal, may be used for extracting the crack skeletons.

A profile extraction method, a boundary tracking method and the like may be used for extracting the crack profiles.

After the cracks are delineated, rasterization processing is performed by using the crack skeletons as a center and the crack profiles as a boundary, and the filling mineral identification is performed in each grid.

A method for identifying the minerals in each grid is consistent with the above method for detecting the mineral composition of the surrounding rocks. That is, the minerals are identified by using a mixed pixel decomposition method, including determination of the end member in the spectral information, acquisition of the spectrum feature of each end member, spectrum matching and identification of types of the minerals.

Finally, the identification result of the crack feature and the crack filler is supplemented to obtain a complete crack identification result. The crack identification result includes the positions of the cracks, the attitudes of the cracks, the apertures of the cracks and the crack fillers.

Under the effect of long-time weathering, a structure of a surface of a rock body of a weathering rock body may change, the cracks may develop, the degree of crushing may increase, the surface mineral composition may change, the quantity of clay minerals or other secondary minerals may increase, the quantity of original minerals may decrease, the corresponding spectrum feature may also change, and the color of the surface of the rock body may also change. For example, under long-time action of carbon dioxide in the air on hydroxides such as calcium hydroxide and magnesium hydroxide in the surface rock body, the color of the surface of the rock body becomes white, and the color representation is more obvious if the degree of weathering is greater. That is, different colors of the surfaces of regions with different degrees of weathering lead to great differences in color differences between the regions at different degrees of weathering and a reference.

Therefore, based on the above analysis, in the present example, the degree of crushing of the surrounding rocks is obtained through the number and the relative areas of the cracks, the mineral weathering variation proportion is obtained by analyzing the stratum lithology and the existing types and contents of the minerals, and the degree of weathering of the surrounding rocks is judged according to the color differences of different weathering regions.

Specifically, the quantity and relative areas of the cracks are calculated according to the crack identification result, the degree of crushing of the rock body is expressed, and a pretrained surrounding rock crushing degree classification model is used for the number and relative areas of the cracks, so as to obtain a classification result for the degree of crushing of the rocks of the weathering surrounding rocks.

Whether there is mineral composition change or not is analyzed according to the stratum lithology, and the existing types and contents of the minerals of the surrounding rocks, the mineral weathering variation proportion is calculated to express the surrounding rock weathering mineral change, and the pretrained mineral weathering variation proportion model is used for obtaining the surrounding rock weathering mineral classification result.

Color information of regions with different types of weathering and degrees of weathering is reflected by using the image spectrum data of each pixel point. According to color features of different regions, a color difference between each pixel point and a control point is calculated, the degree of weathering of each pixel point is evaluated, and a pretrained color difference classification model for regions with different types of weathering and degrees of weathering is used for obtaining a surrounding rock weathering color difference classification result to effectively express different types of weathering surfaces and areas.

Feature vectors of the crushing classification result, mineral classification result, and color difference classification result for representing the weathering of the surrounding rocks are extracted, and are input into a merging analysis module to be respectively weighted to obtain a final surrounding rock weathering degree identification result.

As an optional implementation, for the spectrum-based color measurement, color space linear transformation is performed on reflection spectra of regions with different types of weathering and degrees of weathering, and spectral color difference values between different types of weathering and degrees of weathering are calculated by using a control point as a standard.

As an optional implementation, the relative area is the crack area/target rocklike area, and the crack area may be obtained through calculation on the crack region by using a region growth algorithm.

As an optional implementation, each trained classification model is constructed according to collected rock images of different levels of weathering by establishing mapping relationships of a crack feature-rock body crushing degree, mineral composition variation proportion-weathering degree, and color difference-weathering degree, and the corresponding classification model is continuously updated and optimized as data of tunnel scanning identification and classification accumulates.

As an optional implementation, different surrounding rock weathering levels are divided according to a weathering degree grading table, the degree of weathering of the surrounding rocks is classified according to conditions such as a damage degree of tissue structures, whether the mineral composition changes or not, and color change, and the degree of weathering includes including non-weathering, micro-weathering, medium-weathering, strong-weathering, and full-weathering.

A determining feature of the non-weathering level is fresh rock without weathering.

An identification feature of micro-weathering is that a tissue structure is basically unchanged, only a joint surface has iron-manganese rendering or slight mineral discoloration, and there are few weathering cracks.

A feature of medium-weathering is that a part of a tissue structure is damaged, the mineral composition changes, the minerals attached to the joint surface weather into soil, and weathering cracks develop to pass through the rock body.

A feature of strong-weathering is that most of the tissue structure is damaged, the mineral composition obviously changes, a great number of clay-state clay minerals are contained, and the weathering cracks are prosperously developed to cut the rock body into fragments.

A condition of that the tissue structure is totally damaged, and the mineral composition totally changes and weather into soil is called as full-weathering.

In the present example, a plurality of classifiers obtained by training the above different features are respectively weighted to perform decision-level merging on the weathering degree identification result, and a final surrounding rock weathering degree identification result is finally obtained through joint action.

In the present example, the water outflow form and the water outflow position of the tunnel face to be subjected to detection are identified through image information, and the water outflow form includes no water outflow sign, water seepage, water dripping, threadlike water flowing, water spouting, and water gushing.

Specifically:
the image information is subjected to data enhancement processing. The data enhancement processing includes random fuzzy processing, local magnification, random horizontal reversion, Gaussian sampling and channel scaling, to alleviate a problem of imbalance between tunnel face images with different water outflow volumes, the smooth denoising and detail removal in an image block are realized, and an image edge is reserved to the maximum degree.

A water outflow form of the tunnel face is obtained according to the processed image information by using a trained water outflow image identification model, and a water outflow position is determined. The water outflow image identification model is obtained by training, verifying and testing a built network framework by using the tunnel face images at different water outflow types according to the relationship between the tunnel face water outflow image and the rock water outflow condition of the surrounding rocks of the tunnel face.

In the present example, a digitalized tunnel face geological sketch map is formed according to the engineering geological information and the hydrogeological information of the tunnel face to be subjected to detection obtained in the above process, the output tunnel face geological sketch map includes the digitalized geological information and the tunnel face geological sketch map.

The digitalized geological information is used for briefly describing the engineering geological information and hydrogeological information of the tunnel face, and specifically includes “a mileage, types and content distribution conditions of the minerals of the surrounding rocks, stratum lithology, a crack development degree and crack fillers, forms and distribution of the cracks, the degree of weathering of the surrounding rocks, the water-rich degree of the rock body, and the water outflow mileage, position and water outflow form in the tunnel”.

The tunnel face geological sketch map includes the stratum lithology distribution condition, the distribution and content condition of the minerals of the surrounding rocks, the crack information and the water outflow information. Information obtained after a specific objective is observed by a plurality of different types of hydrogeological information sensors is locally processed on corresponding sensors to form respective geological sketch maps. Images with the same scenario and complementary information are merged, and merging decision is performed to form an image with a richer information amount.

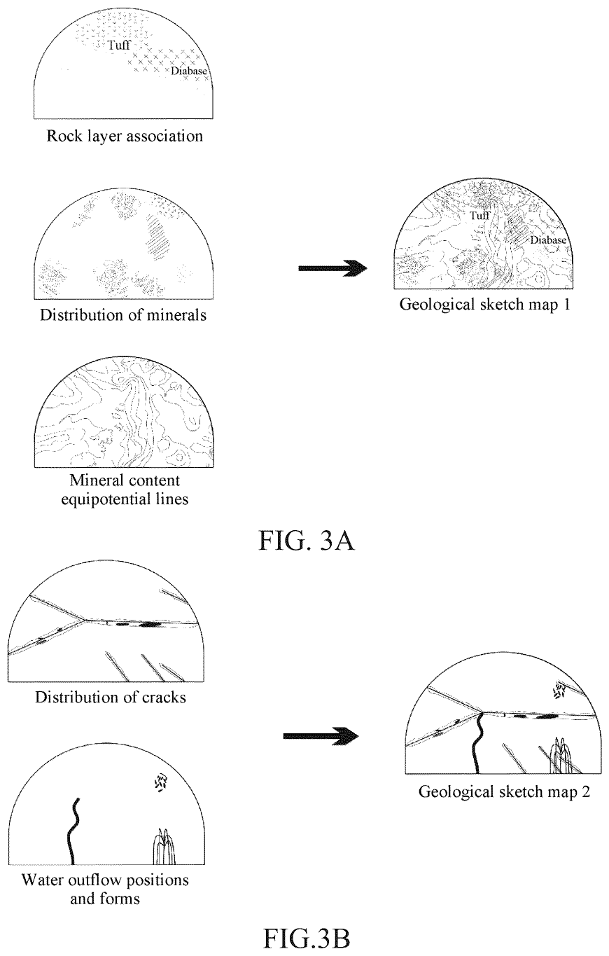

As shown in FIG. 3A to FIG. 3B, in the tunnel face geological sketch map, for the types, space distribution and contents of the minerals: for the types of the minerals, different colors are used for expressing different types of the mines; for the contents of the minerals, the content difference of each type of the minerals in different positions is reflected by content contour lines, and similarly, different minerals are expressed by contour lines of different colors; for example, the distribution of the mineral I is labeled by using a color 1, and the distribution of the mineral II is labeled by using a color 2, and so on; a content value of the mineral I in different positions is analyzed as Z₁, a content value of the mineral II is Z₂, and so on; at the same time, a line color of the content contour line of the mineral I is the color 1, and a line color of the content contour line of the mineral II is the color 2, and so on; and all points of the content values of the same type of the minerals are connected to form a curve, and the content contour line may reflect a content change condition of a specific mineral.

For the stratum lithology, different lithologies are labeled by using the geological lithology legends and symbols; for example, granite is filled with “+”, shale is filled with “−”, and diabase is filled with “X”;
for different degrees of weathering of the surrounding rocks of the tunnel face, if there is a weathering region, the degree of weathering is labeled at the weathering position; and
the mineral identification result is superimposed onto the lithology classification result, and finally, the engineering geological information representing the tunnel face, i.e., the symbols and marks for the stratum lithology, the space distribution and contents of the minerals, and the degree of weathering of the surrounding rocks are merged onto the first geological sketch map.

For the labeling of the crack identification result, according to the identified crack skeletons, profiles and fillers, the positions, attitudes, and apertures of the cracks are labeled by using lines, and the types of the crack filler minerals are labeled by using different colors. For the hydrogeological information of the tunnel face, the water outflow form is expressed by using lines and the like. Therefore, the labeling of information expressing the tunnel face crack features and hydrological features is summarized onto the second geological sketch map.

### Example 2

The present example provides a geological sketching system for tunnel faces based on an image spectrum technology, including:
an image and spectral data body acquiring module, configured to acquire image information and spectral information of a tunnel face to be subjected to detection;
a mineral composition identification module, configured to perform mineral end member extraction and spectral unmixing on the spectral information by using a mixed pixel decomposition method, identify the types and contents of minerals, and determine space distribution of different minerals;
a stratum lithology identification module, configured to extract a spectrum feature vector according to the spectral information, extract an image feature vector according to the image information, and determine the stratum lithology according to the spectrum feature vector and the image feature vector by using a trained classifier;
a crack identification module, configured to extract a crack feature according to the image information, identify a crack filler according to the spectral information, and obtain a crack identification result according to the crack feature and the crack filler;
a weathering degree identification module, configured to acquire the number and relative areas of cracks according to the crack identification result so as to obtain the degree of crushing of surrounding rocks, obtain the weathering variation proportion of the minerals according to the stratum lithology and comparison with existing types and contents of minerals, obtain a color difference classification result of different weathering types and regions according to color features of different regions, and obtain the degree of weathering of the surrounding rocks according to the degree of crushing of the surrounding rocks, the weathering variation proportion of the minerals and the color difference classification result;
a water outflow identification module, configured to identify a water outflow form and a water outflow position of the tunnel face to be subjected to detection according to the image information; and
a geological sketching module, configured to integrate the stratum lithology, the space distribution and contents of the minerals and the degree of weathering of the surrounding rocks into a first geological sketch map, and integrate the crack identification result, the water outflow form and the water outflow position into a second geological sketch map, so as to complete the geological sketch on the tunnel face.

It should be noted herein that the foregoing modules correspond to the steps in Example 1, and examples and application scenarios implemented by the foregoing modules are the same as those implemented by the corresponding steps, but are not limited to the contents disclosed in Example 1. It should be noted that as a part of the system, the foregoing modules may be executed in, for example, a computer system having a group of computer executable instructions.

More examples further provide:
an electronic device, including a memory, a processor, and computer instructions stored in the memory and running on the processor. When the computer instructions are run by the processor, the method in Example 1 is implemented. For brevity, details are not described herein again.

It should be understood that in the present example, the processor may be a central processing unit (CPU), or the processor may be another general-purpose processor, a digital signal processor (DSP), an application-specific integrated circuit (ASIC), a field programmable gate array (FPGA), or another programmable logic device, a discrete gate, a transistor logic device, a discrete hardware component and the like. The general-purpose processor may be a microprocessor, or the processor may be any conventional processor and the like.

The memory may include a read-only memory and a random access memory, and provide an instruction and data to the processor. A part of the memory may further include a non-volatile random access memory. For example, the memory may further store information about a device type.

A computer readable storage medium is used for storing computer instructions. When the computer instructions are executed by the processor, the method in Example 1 is implemented.

The method in Example 1 may be directly reflected to be executed by a hardware processor, or executed by a hardware and software module combination in the processor. A software module may be located in a mature storage medium in the art, such as a random access memory, a flash memory, a read-only memory, a programmable read only memory, an electrically erasable programmable memory, or a register. The storage medium is located in the memory. The processor reads information in the memory and completes the steps of the methods in combination with hardware thereof. To avoid repetition, details are not described herein again.

A person of ordinary skill in the art may be aware that, with reference to the examples described in the present example, units, i.e., algorithm steps, may be implemented by electronic hardware or a combination of computer software and electronic hardware. Whether the functions are executed in a mode of hardware or software depends on particular applications and design constraint conditions of the technical solutions. Technical professionals may use different methods for each particular application to achieve the described functions, but such implementation should not be considered beyond the scope of this application.

The specific implementations of the present disclosure are described above with reference to the accompanying drawings, but are not intended to limit the protection scope of the present disclosure. A person skilled in the art should understand that various modifications or deformations may be made without creative efforts based on the technical solutions of the present disclosure, and such modifications or deformations shall fall within the protection scope of the present disclosure.

1. A geological sketching method for tunnel faces based on an image spectrum technology, comprising:
acquiring image information and spectral information of a tunnel face to be subjected to detection;
performing mineral end member extraction and spectral unmixing on the spectral information by using a mixed pixel decomposition method, identifying the types and contents of minerals, and determining space distribution of different minerals;
extracting a spectrum feature vector according to the spectral information, extracting an image feature vector according to the image information, and determining the stratum lithology according to the spectrum feature vector and the image feature vector by using a trained classifier;
extracting a crack feature according to the image information, identifying a crack filler according to the spectral information, and obtaining a crack identification result according to the crack feature and the crack filler;
acquiring the number and relative areas of cracks according to the crack identification result so as to obtain the degree of crushing of surrounding rocks, obtaining the weathering variation proportion of the minerals according to the stratum lithology and comparison with existing types and contents of minerals, obtaining a color difference classification result of different weathering types and regions according to color features of different regions, and obtaining the degree of weathering of the surrounding rocks according to the degree of crushing of the surrounding rocks, the weathering variation proportion of the minerals and the color difference classification result;
according to the image information, identifying a water outflow form and a water outflow position of a tunnel face to be subjected to detection; and
integrating the stratum lithology, the space distribution and contents of the minerals and the degree of weathering of the surrounding rocks into a first geological sketch map, and integrating the crack identification result, the water outflow form and the water outflow position into a second geological sketch map, so as to complete the geological sketch on the tunnel face.

2. The geological sketching method for tunnel faces based on an image spectrum technology according to claim 1, wherein a process of identifying the types and contents of minerals comprises:
extracting a mineral end member, and performing spectrum matching on the mineral end member and the corresponding spectrum with a prebuilt reference wave spectrum, so as to determine the types of the minerals; and
decomposing the identified types of the minerals as the end member to obtain the percentage contents of each type of minerals, labeling a mineral association, the mineral end member and the contents thereof according to each pixel, and performing weighting processing on the weight of each type of minerals obtained by unmixing each pixel end member and the pixel point occupied by the minerals to obtain the mineral content.

3. The geological sketching method for tunnel faces based on an image spectrum technology according to claim 1, wherein the image information and the spectral information of the tunnel face to be subjected to detection are rasterized, and a spectrum feature vector is extracted after the equalization processing on the spectral information in grids; and
the image feature vector comprises texture features of a feature waveband, and a process of extracting the texture feature comprises: obtaining a two-dimensional gray level image after performing grey processing on the image information, extracting the texture feature of the two-dimensional gray level image by using a grey-level co-occurrence matrix method, and calculating feature parameters in four directions of 0°, 45°, 90°, and 135°.

4. The geological sketching method for tunnel faces based on an image spectrum technology according to claim 1, wherein the crack identification result comprises positions of the cracks, attitudes of the cracks, apertures of the cracks and crack fillers; and
a crack identification process comprises: distinguishing cracks from a background by using an image segmentation method, removing other elements except for the cracks, and acquiring crack skeletons and crack profiles; and performing rasterization processing by using the crack skeletons as a center and the crack profiles as a boundary, and identifying the crack filler according to the spectral information in the grids.

5. The geological sketching method for tunnel faces based on an image spectrum technology according to claim 1, wherein a process of identifying the degree of weathering of the surrounding rocks comprises:
according to the number and relative areas of the cracks, using a pretrained surrounding rock crushing degree classification model to obtain a classification result for the degree of crushing of the surrounding rocks;
analyzing whether there is a mineral composition change or not according to the stratum lithology and comparison with existing types and contents of minerals, and using a pretrained mineral weathering variation proportion model to obtain a mineral weathering variation proportion;
expressing surface color differences of regions of different weathering types and weathering degrees by using spectral color differences according to the color features of different regions; and
respectively weighting the degree of crushing of the surrounding rocks, the mineral weathering variation proportion and the color difference classification result to obtain the degree of weathering of the surrounding rocks.

6. The geological sketching method for tunnel faces based on an image spectrum technology according to claim 1, wherein a water outflow form of the tunnel face is obtained according to the image information by using a trained water outflow image identification model, and a water outflow position is determined;
the water outflow image identification model is obtained by training a built network framework by using the tunnel face images at different water outflow types according to the relationship between the tunnel face water outflow image and the rock water outflow condition of the surrounding rocks of the tunnel face; and
the water outflow form comprises no water outflow sign, water seepage, water dripping, threadlike water flowing, water spouting, and water gushing.

7. The geological sketching method for tunnel faces based on an image spectrum technology according to claim 1, wherein a tunnel face geological sketching process comprises:
labeling the types of the minerals by using different colors;
labeling the contents of the minerals by using content contour lines;
labeling the stratum lithology by using different lithology legends and symbols;
labeling the degree of weathering of the surrounding rocks for a region with surrounding rock weathering;
labeling the positions, the attitudes and the apertures of the cracks by using lines;
labeling the type of the crack filler by using different colors; and
labeling the water outflow form by using lines and the like.

8. A geological sketching system for tunnel faces based on an image spectrum technology, comprising:
an image and spectral data body acquiring module, configured to acquire image information and spectral information of a tunnel face to be subjected to detection;
a mineral composition identification module, configured to perform mineral end member extraction and spectral unmixing on the spectral information by using a mixed pixel decomposition method, identify the types and contents of minerals, and determine space distribution of different minerals;
a stratum lithology identification module, configured to extract a spectrum feature vector according to the spectral information, extract an image feature vector according to the image information, and determine the stratum lithology according to the spectrum feature vector and the image feature vector by using a trained classifier;
a crack identification module, configured to extract a crack feature according to the image information, identify a crack filler according to the spectral information, and obtain a crack identification result according to the crack feature and the crack filler;
a weathering degree identification module, configured to acquire the number and relative areas of cracks according to the crack identification result so as to obtain the degree of crushing of surrounding rocks, obtain the weathering variation proportion of the minerals according to the stratum lithology and comparison with existing types and contents of minerals, obtain a color difference classification result of different weathering types and regions according to color features of different regions, and obtain the degree of weathering of the surrounding rocks according to the degree of crushing of the surrounding rocks, the weathering variation proportion of the minerals and the color difference classification result;
a water outflow identification module, configured to identify a water outflow form and a water outflow position of the tunnel face to be subjected to detection according to the image information; and
a geological sketching module, configured to integrate the stratum lithology, the space distribution and contents of the minerals and the degree of weathering of the surrounding rocks into a first geological sketch map, and integrate the crack identification result, the water outflow form and the water outflow position into a second geological sketch map, so as to complete the geological sketch on the tunnel face.

9. An electronic device, comprising a memory, a processor, and computer instructions stored in the memory and running on the processor, wherein when the computer instructions are run by the processor, the method according to claim 1 is implemented.

10. A computer readable storage medium, configured to store computer instructions, wherein when the computer instructions are executed by the processor, the method according to claim 1 is implemented.
